(19) United States
(12) Patent Application Publication
(10) Pub. No.: US 2024/0002949 A1
(43) Pub. Date: Jan. 4, 2024

(54) PANEL OF MIRNA BIOMARKERS FOR DIAGNOSIS OF OVARIAN CANCER, METHOD FOR IN VITRO DIAGNOSIS OF OVARIAN CANCER, USES OF PANEL OF MIRNA BIOMARKERS FOR IN VITRO DIAGNOSIS OF OVARIAN CANCER AND TEST FOR IN VITRO DIAGNOSIS OF OVARIAN CANCER

(71) Applicant: UNIWERSYTET MEDYCZNY W BIALYMSTOKU, Bialystok (PL)

(72) Inventors: Magdalena NIEMIRA, Bialystok (PL); Anna EROL, Dobra (PL); Jacek Adam KRETOWSKI, Bialystok (PL)

(73) Assignee: UNIWERSYTET MEDYCZNY W BIALYMSTOKU, Bialystok (PL)

(21) Appl. No.: 18/252,634

(22) PCT Filed: Nov. 11, 2020
(86) PCT No.: PCT/PL21/50079

§ 371 Date: May 11, 2023

(30) Foreign Application Priority Data
Nov. 12, 2020 (PL) P.435967

(51) Int. Cl. C12Q 1/6886

(52) U.S. Cl. CPC C12Q 1/6886; C12Q 2600/178; C12Q 2600/158

(57) ABSTRACT

The invention relates to a panel of miRNA biomarkers for in vitro diagnosis of ovarian cancer, a method for in vitro diagnosis of ovarian cancer, uses of a panel of miRNA biomarkers for in vitro diagnosis of ovarian cancer for use in an in vitro diagnostic screening assay for the presence of ovarian cancer, for assessing the effectiveness of ovarian cancer treatment, for monitoring response to ovarian cancer treatment and for predicting recurrence of ovarian cancer after a completed ovarian cancer treatment, as well as a test for in vitro diagnosis of ovarian cancer.

The subject matter of the invention relates to a panel of miRNA biomarkers for diagnosis of ovarian cancer, a method for in vitro diagnosis of ovarian cancer, uses of a panel of miRNA biomarkers for in vitro diagnosis of ovarian cancer and a test for in vitro diagnosis of ovarian cancer.

### TECHNICAL FIELD

The invention generally relates to clinical molecular diagnosis, more specifically to the clinical molecular diagnosis of ovarian cancer, including a non-invasive diagnostic test, the so-called liquid biopsy, of high sensitivity and specificity, in particular for the early diagnosis of ovarian cancer by measuring, analyzing and/or monitoring of the expression of micro RNA (also referred to herein as miRNA) in biological samples, such as blood serum, in particular with the use of a diagnostic classification model.

### BACKGROUND ART

Ovarian cancer is one of the most frequent malignant neoplasms of female sexual organs and the main cause of mortality due to this kind of cancers in developed countries. Currently, the diagnosis of ovarian cancer is based on performing a bimanual pelvic examination, determination of the concentration of CA125 antigen and a transvaginal ultrasound examination. However, it has been estimated that a physical examination performed in women without clinical symptoms makes it possible to detect as few as 1 in 10000 ovarian cancers. Radioimmunoassay for the cancer specific CA125 antigen reveals an increased concentration thereof in 80% of patients suffering from ovarian cancer; however, the concentration may also be higher in the course of non-neoplastic diseases, which limits specificity of the test. Ultrasound examination is not only costly but is also characterized by limited specificity and sensitivity. Apart from that, the main problem in the diagnosis of ovarian cancer is frequently the total lack of symptoms at the early stage, whereas at later stages when metastases already appear the symptoms are often non-characteristic and are associated with the digestive system; that is why diagnostic screening tests are extremely important. Despite numerous studies, there is still a lack of reliable diagnostic biomarkers and methods for early detection of this disease, as well as means for monitoring treatment and/or progression thereof, including early detection of possible recurrence. The diagnostic value of widely used transvaginal ultrasound and determination of the CA125 antigen in serum has proved to be insufficient because of too low sensitivity and specificity.

Currently, the U.S. Food & Drug Administration (FDA) does not recommend screening tests as such for the early diagnosis of ovarian cancer and warns against the tests that have existed so far because, according to FDA, they are not reliable and may mislead both patients and doctors due to a high percentage of false negative or false positive results. At the same time FDA emphasizes that, in the case of other neoplasms, there are effective screening tests but there are no such tests in the case of ovarian cancer, yet. More importantly, in the said statement FDA clearly emphasizes that a good screening test in the case of ovarian cancer is highly needed due to the fact that the diagnosis is usually too late. Scientists have even been given an easy access to a biobank with blood samples of patients with ovarian cancer in order to accelerate research for an efficacious diagnostic biomarker and method for diagnosing ovarian cancer, in particular at the early stage thereof. Studies aimed at developing a sensitive and specific test based on non-invasive biomarkers are therefore urgently needed in the diagnosis of ovarian cancer. Due to the fact that the ovaries are organs that lie entirely within the peritoneal cavity, it is currently impossible to diagnose ovarian cancer without surgical resection. Additionally, due to the possibility of easy dissemination of cancer cells, thin-needle biopsy should also be avoided. Therefore, there is an urgent need for non-invasive biomarkers which could support methods used so far, such as transvaginal ultrasound and measurement of CA125 level (a marker with merely 40% sensitivity).

It is known that micro RNA (miRNA) expression may be found in a cancer tissue to be present at a different level than in a normal tissue. Various miRNAs are known and the use of expression analysis of many different microRNAs for the diagnosis of various types of cancers is also known. miR-1246 is used for the diagnosis of lung, oral cavity, uterine cervix or prostate cancers (see e.g. Liao et al., Expression and Clinical Significance of microRNA-1246 in Human Oral Squamous Cell Carcinoma (2015) https://www.ncbi.nlm.nih.gov/pmc/articles/PMC4371709/, Zhang et al., Tumour-initiating cell-specific miR-1246 and miR-1290 expression converge to promote non-small cell lung cancer progression, Nature Communications (2016)). miR-1246 sequence is available in databases, e.g. www.mirbase.org under the accession number MIMAT0005898. It is also known that the expression of this miRNA is changed in the serum of patients with stomach cancer. However, despite the fact that miR-1246 expression may be changed in the serum of patients with various types of neoplasms, such phenomenon has not been demonstrated in the case of ovarian cancer. One should also bear in mind that the fact that given type of miRNA molecule is found at the changed level in patients with one type of cancer does not exclude the possibility that such dependence will not be found in the case of cancers of a different type, or that it is only in an appropriate combination with another biomarker or biomarkers that its expression may possibly provide a diagnostic indicator and measurement thereof may be part of an efficacious non-invasive test for a different, specific cancer, e.g. ovarian cancer.

MicroRNA 150-5p (miR-150-5p) is also known. The miRNA sequence is available in databases, e.g. www.mirbase.org under the accession number: MIMAT0000451. It is known that miR-150-5p is involved in the formation of numerous cancers. For example, a reduced level of the miR-150-5p expression was detected in tissue samples (but not in the blood or serum) from patients with pancreatic cancer as compared to healthy tissues. (Zhonghua Bing Li Xue Za Zhi., July 2013; 42(7):460-4. doi: 10.3760/cma.j.issn.0529-5807.2013.07.007.). However, so far no relationship has been demonstrated between the expression of miR-150-5p and ovarian cancer, either alone or in combination with any other biomarker or biomarkers.

The object of the invention is therefore to provide a sensitive and specific diagnostic and prognostic biomarker for the diagnosis of ovarian cancer and monitoring of its treatment, as well as for predicting its recurrence after a completed treatment. The object of the invention is also to provide a non-invasive method for the diagnosis of ovarian cancer which is characterized by a high specificity and sensitivity, and a diagnostic test appropriate for diagnostic assays, including screening assay for this cancer.

### SUMMARY OF INVENTION

The subject matter of the invention relates to a panel of miRNA biomarkers comprising miR-1246 and miR-150-5p. Such a panel of miRNA biomarkers is intended for use in the diagnosis of ovarian cancer.

Preferably, the panel of miRNA biomarkers according to the invention consists of miRNA biomarkers: miR-1246 and miR-150-5p.

The subject matter of the invention relates to a method for in vitro diagnosis of ovarian cancer in a subject, consisting in that it comprises the following steps:
i) determining the expression level of a panel of two miRNA biomarkers: miR-1246 and miR-150-5p in a sample from a subject;
ii) comparing the levels determined in step i) with the expression levels of miR-1246 and miR-150-5p in a subject without ovarian cancer, wherein the comparison provides a diagnostic indicator determining whether the subject has ovarian cancer.

Preferably, in the method according to the invention an increase in the expression miR-1246 level relative to the miR-1246 expression level in a subject without ovarian cancer and a decrease in the miR-150-5p expression level relative to the miR-150-5p expression level in a subject without ovarian cancer indicate ovarian cancer in the subject. In other words, the diagnostic indicator of ovarian cancer is an increase in the miR-1246 expression level in the tested sample relative to the miR-1246 expression level in a subject without ovarian cancer and a decrease in the miR-150-5p expression level in the tested sample relative to the miR-150-5p expression level in a subject without ovarian cancer.

Preferably, in the method according to the invention the comparison of expression levels is carried out with the use of a data set comprising data on the expression level of a panel of miRNA markers comprising miR-1246 and miR-150-5p.

Preferably, in the method according to the invention the expression level of a panel of miRNA biomarkers is determined with the use of a method for measuring expression selected from reverse transcription—quantitative real-time polymerase chain reaction (RT-qPCR), NanoString or microarray method.

Preferably, in the method according to the invention expression levels are compared after normalization.

Preferably, in the method according to the invention a diagnostic classification model is used which, on the basis of data concerning the level of miR-1246 and miR-150-5p, classifies a sample as a sample from a subject with ovarian cancer or as a sample from a subject without ovarian cancer. Such a diagnostic classification model uses optimal cut-off points depending on the applied method for determining the expression level of a panel of miRNA biomarkers, i.e. NanoString, microarray, RT-qPCR, with emphasis on RT-qPCR. The preferable method according to the invention always uses an optimal cut-off point for the applied method for determining the expression level of a panel of miRNA biomarkers and a range with information about specificity (Sp) and sensitivity (S). A diagnostic classification model always uses an optimal cut-off point within the range of 0.1-0.8 for the results of measurement of the expression of the panel of miRNA biomarkers according to the invention carried out with the use of RT-qPCR method; in the case of NanoString method a cut-off point is within the range of 0.3-09, and in the case of microarray method the value of an optimal cut-off point is 0.5.

More preferably, RT-qPCR method is used for determining the expression level of miRNA. This method is suitable for use on a large scale in diagnostic laboratories, because it is much more cost-effective than NanoString or microarray methods, and additionally, it requires much less test material and makes it possible to obtain results much faster than in the case of large-scale study methods, such as NanoString or microarrays.

Even more preferably, in the case of expression measurement with the use of RT-qPCR method, the expression level of a reference miRNA, preferably miR-103-3p and/or miR-199b-5p is used to normalize the results.

Even more preferably, normalization of results is obtained with the use of the following formula:

deltaCt=(Ct miR-1246/miR-150-5p-Ct miR-103-3p),
wherein
delta Ct is a change in the value of the threshold cycle
Ct is the value of the threshold cycle.

Most preferably, the obtained data in the form of deltaCt values are substituted into a diagnostic classification model and the obtained result is compared with a cut-off point selected from the range of 0.1-0.8. The value of this point is determined by training the diagnostic classification model using data of the known status samples.

Importantly, in this preferable method according to the invention deltaCt values are not compared, but deltaCt values are substituted into the above equation and they are compared with the cut-off point determined by the diagnostic classification model.

Preferably, in the method according to the invention a serum sample is used as a sample from a subject.

Preferably, a panel of biomarkers consisting of miR-1246 and miR-150-5p is used for the diagnosis, especially of high-grade serous ovarian cancer.

The subject matter of invention also relates to the use of the panel of miRNA biomarkers as defined above, i.e. a panel comprising or preferably consisting of miR-1246 and miR-150-5p, for in vitro diagnosis of ovarian cancer.

The subject matter of the invention also relates to the use of the panel of miRNA biomarkers as defined above in in vitro diagnostic screening assay for the presence of ovarian cancer.

The subject matter of the invention also relates to the use of the panel of miRNA biomarkers as defined above for in vitro assessment of the effectiveness of ovarian cancer treatment. The subject matter of the invention also relates to the use of the panel of miRNA biomarkers as defined for in vitro monitoring of the response to ovarian cancer treatment.

The subject matter of the invention further relates to the use of the panel of miRNA biomarkers as defined above for predicting recurrence of ovarian cancer after a completed ovarian cancer treatment.

The subject matter of the invention further relates to a test for in vitro diagnosis of ovarian cancer, characterized in that it comprises means for quantitative determination of the expression level of a panel of two miRNA biomarkers: miR-1246 and miR-150-5p and instructions for carrying out the method according to the invention as defined above.

Preferably, the diagnostic test according to the invention comprises reactants and primers for amplification in RT-qPCR reaction of miR-1246 and miR-150-5p as means for quantitative determination of the expressions of the panel of miRNA biomarkers: miR-1246 and miR-150-5p.

More preferably, the diagnostic test according to the invention further comprises means for quantitative determination of expression of a reference miRNA, preferably, the panel of miR-103-3p and/or miR-199b-5p, such as starters and reactants appropriate for amplification by RT-qPCR method.

### DETAILED DESCRIPTION OF INVENTION

The inventions according to the application are based on the selection by the inventors of a panel of mikroRNA biomarkers, i.e. miR-1246 and miR-150-5p, which is suitable for the diagnosis of ovarian cancer, in particular high-grade serous ovarian cancer, with high sensitivity and specificity.

The inventions according to the application solve the problem of the present lack of reliable, sensitive and specific biomarkers and diagnostic methods using them for the diagnosis of ovarian cancer, in particular high-grade serous ovarian cancer, and consequently enable the diagnosis of this neoplastic disease, preferably even an early diagnosis of ovarian cancer, i.e. before the appearance of clinical symptoms of this disease. Additionally, the results of diagnostic classification obtained in accordance with the present inventions are stable also with respect to progression of the disease, which fact has been confirmed by external data. This feature of the inventions according to the present application enables their wider diagnostic application, in particular in a screening assay for the presence of ovarian cancer. The panel of miRNA biomarkers according to the invention, comprising miR-1246 and miR-150-5p and preferably consisting of such miRNA biomarkers, enables a specific and sensitive diagnosis of ovarian cancer, especially high-grade serous ovarian cancer.

The panel of miRNA biomarkers according to the invention is also applicable in accordance with the invention in an in vitro screening assay for the presence of ovarian cancer.

The panel of miRNA biomarkers according to the invention is also applicable in accordance with the invention in a method for the assessment of efficacy of in vitro ovarian cancer treatment.

The panel of miRNA biomarkers according to the invention is also applicable in accordance with the invention for the monitoring of the response to ovarian cancer treatment.

The panel of miRNA biomarkers according to the invention is also applicable in accordance with the invention for predicting recurrence of ovarian cancer after a completed ovarian cancer treatment.

Preferably, in such applications of the panel of miRNA biomarkers according to the invention as defined above, the panel consists of miR-1246 and miR-150-5p.

Thus, the inventions presented herein may be used not only for the diagnosis of ovarian cancer but also monitoring of the effectiveness of ovarian cancer treatment, both in the course thereof and after completion of the treatment. In such case a sample for tests is taken from a subject during treatment and/or after completed treatment, and, at specific time intervals, the expression level of a panel of miRNA biomarkers in the sample from the patient is determined and is compared with respective data for that patient obtained earlier, i.e. at the stage of diagnosing ovarian cancer and/or at an earlier stage of a treatment. A change in the expression level of a panel of miRNA biomarkers according to the present invention relative to the level determined earlier will provide a diagnostics indicator allowing one to determine whether the applied treatment is effective. A diagnostic indicator of ovarian cancer preferably is an increase in the miR-1246 expression level relative to the miR-1246 expression level in a subject without ovarian cancer and a decrease in the miR-150-5p expression level relative to the miR-150-5p expression level in a subject without ovarian cancer. A change in the expression levels of the panel of miRNA biomarkers according to the invention in the course of treatment towards the expression levels of that panel of biomarkers in subjects without ovarian cancer indicates the effectiveness of ovarian cancer treatment. After a successfully completed treatment, the expression level of a panel of miRNA biomarkers according to the invention should basically be the same as in the subject without ovarian cancer. Thus, the finding of the increased miR-1246 expression level relative to the miR-1246 expression level in the subject without ovarian cancer and the decreased miRNA-150-5p expression level relative to the miR-150-5p expression level in the subject without ovarian cancer after a completed treatment allows one to determine recurrence of ovarian cancer by classifying the tested sample as a sample from a subject with ovarian cancer.

The method for in vitro diagnosis of ovarian cancer according to the invention is not only sensitive and specific but also quick and non-invasive because it only requires a simple blood collection in order to isolate serum.

To carry out the method according to the invention, a sample from a subject is needed. The sample is preferably a serum sample. A whole blood sample is collected from a subject for examination in a standard way to test tubes without an anticoagulant (to obtain a blood clot) and then serum is isolated therefrom for analysis in accordance with the invention. More specifically, after collecting, the blood sample is set aside for 30-60 minutes, and then it is centrifuged at 4000 rpm for 5 min. A serum sample obtained in this way is transferred to an RNase-free test tube in order to carry out the method and/or test according to the invention. Subsequently, from the sample collected in this way from a subject miRNA is isolated with the use of commercially available kits for isolating miRNA, following the instructions of their manufacturers. In the case RNA is isolated from body fluids, such as serum, it is difficult to assess isolation efficiency on the basis of a spectrophotometric measurement because the amount of material is small. That is why a fluorometric method may be used as a method for monitoring the quality and quantity of isolated material. Thus, when carrying out the method according to the invention, after isolation of miRNA preferably the concentration of miRNA is determined using a fluorometric method, e.g. with the use of Qubit™ microRNA Assay Kit and Qubit™ fluorometer (ThermoFisher Scientific, USA). Based on the changed expressions of miRNA molecules in the ovarian cancer group under examination with relation to the group without ovarian cancer, selected by NanoString method, an attribute selection method was used and the strongest candidates were selected to develop a diagnostic classification model and a diagnostic test based on it. Classification models were developed from various combinations of selected miRNAs, among which the panel of miRNA biomarkers according to the invention comprising miRNA: miR-1246 and miR-150-5p proved to be the best, i.e. the most sensitive and specific combination. The use of the panel consisting of these two miRNA biomarkers enables a sensitive and specific diagnosis of ovarian cancer, even at its early stage of development.

Table 1 below shows parameters for the panel of biomarkers according to the invention in comparison with another combination of miRNA biomarkers.

TABLE 1
Variables in developed classification
models with different panels of miRNA.

| | | Coefficient for | Coefficient for |
|---|---|---|---|
| Coefficients | Constant a₀ | predictor x₁ (a₁) | predictor x₂ (a₂) |
| x1 = miR-1246 | 4.47117 | 0.07091 | −0.31985 |
| x2 = miR-150-5p | | | |
| x1 = miR-1246 | −0.94138 | 0.03202 | −0.02179 |
| x2 = miR-144-3p | | | |

Table 2 below shows a comparison of basic quality parameters of a classification for the panel of biomarkers according to the invention in comparison with another combination of miRNA biomarkers. As can be clearly seen, the quality parameters, i.e. area under the curve, specificity and sensitivity, for the panel of biomarkers according to the invention are clearly better than in the case of another set of biomarkers.

TABLE 2
Quality parameters for diagnostic classification models in training
and test sets for expression measurements by NanoString method.

| | Name | | | |
|---|---|---|---|---|
| | miR-1246, | miR-1246, | miR-1246, | miR-1246, |
| | miR-150-5p | miR-150-5p | miR-144-3p | miR-144-3p |
| Set | Training | Test | Training | Test |
| Area under | 98.6% | 100% | 93.9% | 95.2% |
| Curve (AUC) | | | | |
| Confidence | 96.4% | — | 85.8% | 85.2% |
| Interval (CI) | | | | |
| lower limit | | | | |
| CI upper limit | 100% | — | 100% | 100% |
| Cut-off point | 0.44 | 0.44 | 0.56 | 0.56 |
| (Youden index) | | | | |
| Sensitivity | 96.4% | 100% | 92.9% | 92.3% |
| Specificity | 95.2% | 92.3% | 95.2% | 87.5% |
| Set | Training | Test | Training | Test |
| Coefficient of | 0.74 | — | 0.66 | — |
| determination | | | | |
| (R²) | | | | |
| Root Mean | 0.23 | — | 0.29 | — |
| Square | | | | |
| Error (RMSE) | | | | |

The method according to the invention can be carried out with the use of any method for comparing gene expression levels, but it is preferably carried out with the use of diagnostic classification models developed by the present inventors by inserting data, in this case data concerning the expression levels of the panel of microRNA biomarkers according to the invention. Thus, in accordance with the invention, diagnostic classification models have been developed the main role of which is to differentiate cases of ovarian cancer from cases without ovarian cancer by determining and comparing the expression levels of a panel of selected miRNA biomarkers, i.e. miRNA-1246 and miRNA-150-5p. These diagnostic classification models have been adjusted, with the use of a machine learning technique and by determining appropriate cut-off points, to known and generally available methods for assessing gene expression levels as described above. Thus, the method according to the invention can be carried out with the use of known methods for miRNA expression measurements, preferably such as microarray or NanoString platform, and most preferably, quantitative reverse transcription and real-time polymerase chain reaction (RT-qPCR). In order to effectively use the method according to the invention, it is only necessary to determine the expression level of the panel of miRNA biomarkers according to the invention in a sample from a subject, preferably in a serum sample, and normalize the result in accordance with the applied measurement method, as it is known in this art and as described above. Subsequently, in the method according to the invention a comparison of the expression levels is made, preferably by classifying a sample as a sample from a subject with ovarian cancer or as a sample from a subject without ovarian cancer, with the use of a diagnostic classification mode, using a logistic regression model, substituting the result concerning the expression level of selected miRNA from a subject and comparing it with a cut-off point appropriate for the used method for measuring the expression of miRNA biomarkers according to the invention. According to the invention the result for a diagnostic classification model is calculated with the use of a data set comprising data on the expression level of a panel of miRNA biomarkers comprising miR-1246 and miR-150-5p. Due to the fact that a cut-off point is determined on the basis of results of classification in the training set, it is not required to subsequently make any comparison with data from subjects without ovarian cancer, because the classification is based on the comparison of the result of the diagnostic classification model with an optimal cut-off point determined on the basis of the trained diagnostic classification model. The value of an optimal cut-off point is determined at the point of best results for specificity and sensitivity in the course of training the model. This means that a diagnostic classification model is trained on the basis of data input from the training set and it classifies a sample as a sample originating from a subject with ovarian cancer or as a sample from subjects without ovarian cancer.

Importantly, in the method according to the invention deltaCt values preferably are not compared, but these deltaCt values are input into a diagnostic classification model and are compared with a pre-determined optimal cut-off point appropriate for the applied method for measuring the expression of miRNA biomarkers according to the invention.

According to the invention, a diagnostic classification model developed on the basis of normalized data on the miRNA expression in serum on NanoString platform is as follows:

$$P ( Y = 1 \vert {x}_{1} , {x}_{2} , … , {x}_{k} ) = \frac{{e}^{4.47 + 0.071 * miR - 1246 - 0.32 * miR - 150 - 5 p}}{1 + {e}^{4.47 + 0.071 * miR - 1246 - 0.32 * miR - 150 - 5 p}}$$

A cut-off point within the range of 0.3<x<0.9 in this case gives a result for specificity (Sp) and sensitivity (S)>80% (see Table 7 below).

According to the invention, a diagnostic classification model developed on the basis of normalized data on the miRNA expression in serum on a microarray platform (e.g. Affymetrix) is as follows:

$$P ( Y = 1 \vert {x}_{1} , {x}_{2} , … , {x}_{k} ) = \frac{{e}^{- 2.57 + 0.054 * miR - 1246 + 0.49 * miR - 150 - 5 p}}{1 + {e}^{- 2.57 + 0 , 054 * miR - 1246 + 0.49 * miR - 150 - 5 p}}$$

A cut-off point equal to 0.5 in this case gives a result for specificity (Sp) and sensitivity (S)>80% (see Table 11 below).

According to the invention, a diagnostic classification model developed on the basis of normalized data on the miRNA expression in serum by RT-qPCR method is as follows:

$$P ( Y = 1 \vert {x}_{1} , {x}_{2} , … , {x}_{k} ) = \frac{{e}^{55.16 - 1.616 * miR - 1246 + 4.277 * miR - 150 - 5 p}}{1 + {e}^{55.16 - 1.616 * miR - 1246 + 4.277 * miR - 150 - 5 p}}$$

A cut-off point in this case within the range of 0.1<x≤0.8 gives a result for specificity (S) and sensitivity (Sp) in a test set >85% (see Table 16 below).

According to the invention, the measurement of the expression level of the panel of miRNA biomarkers according to the invention is conducted by RT-qPCR method. This method is considered to be accurate, sensitive and specific in the context of mature miRNAs. It enables determining the expression level of miRNAs even with low levels. The test requires a sample of a small volume. The method is preferably carried out in two steps: 1) reverse transcription reaction (RT) and 2) proper polymerase chain reaction (PCR). Both steps are conducted in a standard manner known in this art, with the use of commercially available kits and starters for RT and PCR reactions specific for the two selected miRNAs constituting the panel of miRNA biomarkers according to the invention, i.e. miR-1246 and miR-150-5p, using conditions recommended by the manufacturers thereof. In this method, preferably the expression level of a reference gene, preferably the selected miR-103-3p, is also measured in order to enable normalization of results. Before the beginning of the proper reaction, a series of template dilutions is prepared, for the purpose of calculating the effectiveness of reaction of each pair of starters. Each time the proper reaction is conducted for cDNA obtained with the participation of RNA reverse transcriptase (RT+), as well as for the control samples without the addition of reverse transcriptase (RT−) and for samples/tests with water only instead of an array. When Ct values for each miRNA have been obtained, the results are normalized to the Ct values of the reference gene and a value is calculated according to the following formula:

deltaCt=(Ct miR-1246/miR-150-5p-Ct miR-103-3p).

Data obtained from RT-qPCR reaction in the form of values of difference in expression (deltaCt) are preferably inserted in accordance with the invention to a diagnostic classification model, the result of which, after comparing the result of the equation with a cut-off point, as described above, makes it possible to classify the patient from which the material was taken as a patient with ovarian cancer or without ovarian cancer.

As it is known, it is particularly recommended, when conducting a real-time PCR reaction in the case of miRNA analyses, to calculate the amplification efficiency of applied starters on the basis of results obtained from a series of 10-fold template dilutions. To this end, it is necessary to determine a regression curve on the basis of obtained measurement points and the slope of the line coefficient. In the methods according to the invention the optimization step of PCR method is performed, which optimization step consists in assessing the efficiency of each pair of the starters used, optimizing the concentration of cDNA template and the concentration of applied starters, in a manner known in the art.

The method for the diagnosis of ovarian cancer according to the invention enables a non-invasive diagnosis of ovarian cancer. One advantage of the method according to the invention is that is it characterized by a high diagnostic and/or prognostic sensitivity and specificity. It should be emphasized that the method according to the invention has proved to be effective in the diagnosis of ovarian cancer at its various stages, also at the early stages (FIGO I-II), which are very rarely detected when using currently available methods. Such a method is particularly advantageous economically for health services, because it makes it possible to diagnose ovarian cancer at the early stage of its development and consequently apply effective treatment at an earlier stage and thus increase effectiveness of the treatment, lower the cost of the treatment and improve the quality of patient's life. Another advantage of the invention is that the examination requires only a small amount of blood sample from which serum is isolated for the examination in accordance with the invention and thus it is not necessary to perform an invasive fine needle biopsy which carries a risk to the patient. Due to the fact that the method according to the invention uses a panel of miRNA biomarkers, which is highly precise because it shows a higher AUC, that is sensitivity and specificity, than CA125 marker widely used in the diagnosis of ovarian cancer (on the basis of data from literature); the use of this panel of biomarkers and preferably methods using diagnostic classification models may be recommended by oncologists for a quick confirmation of a preliminary diagnosis. The diagnostic method and test according to the invention may be used in diagnostic laboratories, in screening assays supporting early diagnosis of ovarian cancer, for assessing the effectiveness of ovarian cancer treatment or for monitoring patients after a completed treatment (the so-called follow-up). The inventions according to the application are also capable of application in diagnostic screening assays for the presence of ovarian cancer, e.g. in risk groups such as women aged over 40, in particular those with positive family history of ovarian cancer or breast cancer. Such diagnostic screening tests are particularly advantageous in cases of that kind because currently there are no effective means that would enable the diagnosis of ovarian cancer at early stages of its development, in particular in such populations.

The results obtained according to the invention testify to a high sensitivity and specificity of inventions presented herein and surpass the results obtained with the use of diagnostic means known in the art, in particular those based on the measurement of CA125 marker and transvaginal ultrasound examination.

Now the invention will be illustrated by means of examples and figures, which however are not intended to limit in any manner the scope of protection defined in the claims.

### BRIEF DESCRIPTION OF FIGURES

FIG. 1 shows a heat map with hierarchical clustering for various miRNAs.

FIG. 2 shows ROC curves and AUC (Area Under the Curve) for various miRNAs obtained on the basis of data on the expression level of miRNA molecules with the use of NanoString platform.

FIG. 3 shows ROC curves and AUC (Area Under the Curve) for a training set and a test set for the diagnostic classification model based on data on the miR-1246 and miR-150-5p expression levels obtained with the use of NanoString platform. The graph includes the AUC value, the cut-off point calculated by Youden's J statistic method, and sensitivity and specificity corresponding to that point.

FIG. 4 shows ROC curves and AUC for a training set and a test set for the diagnostic classification model based on public data on the miR-1246 and miR-150-5p expression levels obtained with the use of an array technique, which are included in Gene Expression Omnibus (GEO) database. The graph includes the AUC value, the cut-off point, and sensitivity and specificity corresponding to that point.

FIG. 5 shows ROC curves and AUC for a diagnostic classification model depending on the stage of ovarian cancer (FIGO I-IV). The graph includes the AUC value, the cut-off point, and sensitivity and specificity respectively corresponding to that point.

FIG. 6 shows ROC curves and AUC for the diagnostic classification model depending on the type of cancer, the values in parentheses show the confidence interval. The analysis was conducted with the use of public data on the miRNA expression level obtained by a microarray technique for lung cancer and colorectal cancer.

FIG. 7 shows relative expression level of miR-1246 and miR-150-5p in serum of women with ovarian cancer in comparison to the control group, obtained with the use of RT-qPCR technique.

FIG. 8 shows ROC curves and AUC for a training set and a test set for the diagnostic classification model obtained on the basis of data obtained with the use of RT-qPCR technique. The graph includes the AUC value, and the values in parentheses show the confidence interval.

### EXAMPLES

When carrying out all studies and assays described herein, the inventors used standard, generally known procedures for the preparation of biological material, isolation of miRNA and measurement of the expression level of miRNA, as well as commercially available sets and devices for these purposes, including known software for analysis, acting in accordance with recommendations of the producers thereof, and known statistical and bioinformatics methods, unless clearly indicated otherwise.

The method for the diagnosis of ovarian cancer was developed on the basis of the expression of selected miRNA molecules, first by selecting appropriate molecules with the use of a large-scale NanoString platform, and then by verifying the obtained results in tests based on quantitative DNA polymerase chain reaction (RT-qPCR). All test were approved by the Bioethics Committee of Medical University of Bialystok (approval no. PK.002.69.2020).

Profiling the miRNA Expression in Serum with the Use NanoString Platform

Profiling the miRNA expression in serum with the use NanoString platform was conducted in a group (n=70) comprising patients with high-grade serous ovarian cancer (n=36) and in a control group, i.e. a group of persons without ovarian cancer (n=34). The p-value for age and Body Mass Index (BMI) was calculated by means of the Wilcoxon rank sum test. No statistically significant differences in age and BMI were found between the groups (p-value >0.05). The characteristics of the participants are presented below in Table 3. Blood samples were collected before the beginning of treatment.

TABLE 3
Characteristics of patients whose miRNA profile in serum
was determined with the use of NanoString platform.

| | Control | Ovarian cancer |
|---|---|---|
| Number of patients | 34 | 36 |

| | Mean ± SD | Min | Max | p-value |
|---|---|---|---|---|
| Age at diagnosis | | | | |
| (years) | | | | |
| Healthy | 63 + 14 | 38 | 86 | |
| Diseased | 59 + 6 | 45 | 72 | 0.1 |
| BMI (weight/height²) | | | | |
| Healthy | 29 + 14 | 38 | 86 | 0.4 |
| Diseased | 27 + 3 | 21 | 34 | |

| | FIGO I | FIGO II | FIGO III-IV | N/A |
|---|---|---|---|---|
| Number of cases | 3 | 1 | 31 | 1 |

Experimental Verification of the Invention Comprised the Following Steps:

I. Profiling miRNA in the Group with Ovarian Cancer and in the Control Group

Extraction of RNA from serum was conducted by means of miRCURY™ RNA isolation Kit (Exiqon, Denmark), in accordance with the producer's protocol. In all samples the concentration of RNA was quantitatively determined by a fluorometric method with the use of Qubit™ apparatus (Thermo Fisher Inc., USA). The analysis was made in 6 balanced experiments with the use of NanoString platform. It enables simultaneous detection of the expression of 798 miRNAs in one sample. All steps were conducted according to the producer's protocol. Data were analyzed with the use of nSolver software, version 4.0. Normalization was conducted by means of the geometric mean for Top100 miRNA. Fold change (FC) was calculated by determining the healthy subjects as the baseline level. Correction for multiple hypothesis testing was introduced using the expected False Discovery Rate (FDR), which is the value of expected fraction of false rejections of null hypotheses in the set of all rejected null hypotheses, multiplied by the probability of rejecting at least one hypothesis according to Benjamin-Hochberg. In patients with high-grade serous ovarian cancer, significantly different expression profiles of twelve miRNAs in serum were found as compared with the controls (Table 4). Data in the form of counts after normalization were imported and used in further statistical analysis. In the table, the downward arrow marks miRNAs whose expression is reduced, the upward arrow marks the miRNAs whose expression is elevated in patients with ovarian cancer as compared with the group without ovarian cancer.

TABLE 4
Parameters of fold change and p-value after
Benjamin-Hochberg correction (FDR).

| miRNA | FC | | FDR |
|---|---|---|---|
| miR-144-3p | −2.31 | ↓ | 0.00 |
| miR-142-3p | −1.94 | ↓ | 0.00 |
| miR-150-5p | −1.81 | ↓ | 0.00 |
| miR-15a-5p | −1.75 | ↓ | 0.00 |
| miR-15b-5p | −1.65 | ↓ | 0.00 |
| miR-126-3p | −1.61 | ↓ | 0.00 |
| miR-4454 + | 2.45 | ↓ | 0.00 |
| miR-7975 | | | |
| miR-1246 | 2.61 | ↓ | 0.00 |
| miR-191-5p | −1.51 | ↓ | 0.01 |
| miR-4516 | 1.68 | ↓ | 0.01 |
| miR-630 | 2.16 | ↓ | 0.01 |
| miR-106b-5p | −1.36 | ↓ | 0.04 |

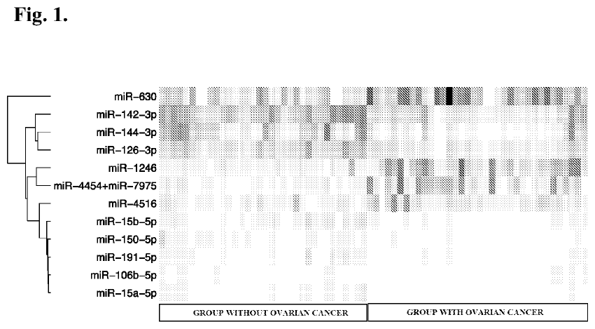

FIG. 1. shows a heat map including 12 miRNAs differentially expressed in patients with ovarian cancer and in the control group, that is without ovarian cancer. Differentially expressed miRNA molecules were hierarchically clustered by means of the Euclidean distance metric with the complete linkage method. The presence of four miRNA clusters was revealed.

II. ROC for Each of the Differentiated miRNAs

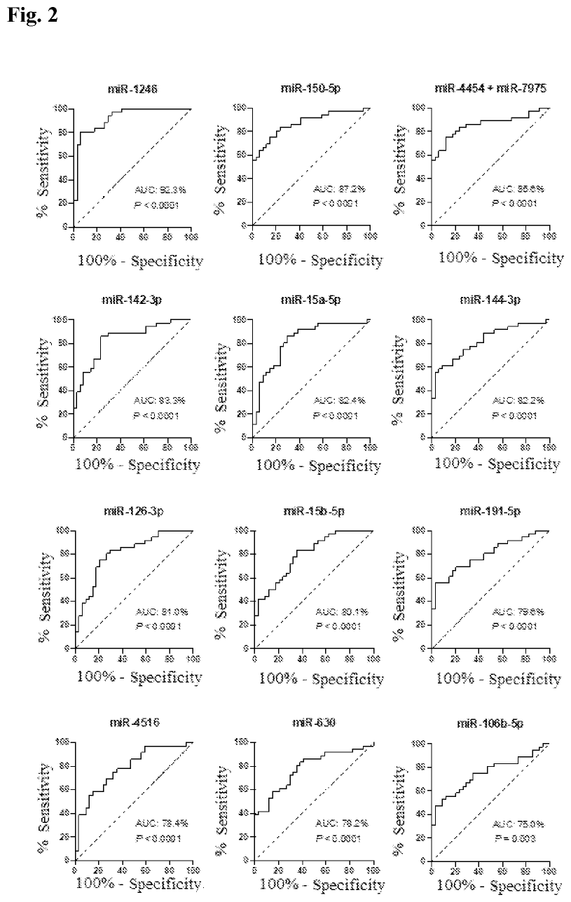

ROC curve makes it possible to assess correctness of a classifier which may prove to be a potential diagnostics marker. It also makes it possible to calculate specificity and sensitivity at a specific cut-off point. Specificity (Sp) is the rate of true negative results, that is subjects who do not have a cancer and are classified as such on the basis of a classifier. Sensitivity (S) is equal to the rate of true positive results, that is subjects who have a cancer and are classified as such on the basis of a classifier. pROC package [X. Robin et al., “pROC: An open-source package for R and S+ to analyze and compare ROC curves,” BMC Bioinformatics, vol. 12, no. 1, p. 77, March 2011] in R [R. C. T. R. Foundation, “R: A Language and Environment for Statistical Computing,” vol. 2, https://www.R-project.org, 2013.] was used to calculate AUCs together with the confidence interval (95%), cut-off point, sensitivity and specificity, and ROC curve was created for each of the differentiated miRNAs. The results are presented in Table 5 and FIG. 2.

TABLE 5
Predictability of differentiated miRNAs. The table presents ROC curve
parameters (Area Under the Curve (AUC) and 95% confidence
interval (CI) and sensitivity (S) and specificity (Sp) corresponding
to the optimal cut-off point selected by Youden's method.

| | | Upper | Lower | | | |
|---|---|---|---|---|---|---|
| Differentiated | AUC | limit | limit | Cut-off | | |
| miRNA | (%) | CI (%) | CI (%) | point | S | Sp |
| miR-1246 | 92.3 | 86.1 | 98.6 | 92.5 | 80.6 | 94.1 |
| miR-144-3p | 82.2 | 72.4 | 92 | 53.0 | 55.6 | 97.1 |
| miR-4454 + | 85.6 | 76.3 | 94.9 | 74.2 | 75 | 88.2 |
| miR-7975 | | | | | | |
| miR-150-5p | 87.2 | 78.8 | 95.5 | 31.1 | 75 | 85.3 |
| miR-630 | 78.2 | 67.3 | 89.1 | 149.8 | 80.6 | 64.7 |
| miR-142-3p | 83.3 | 73.8 | 92.9 | 168.1 | 86.1 | 76.5 |
| miR-15a-5p | 82.4 | 72.4 | 92.5 | 27.79 | 86.1 | 70.6 |
| miR-15b-5p | 80.1 | 70.0 | 90.1 | 44.1 | 83.3 | 64.7 |
| miR-191-5p | 79.6 | 69.1 | 90.1 | 27.2 | 55.6 | 97.1 |
| miR-106b-5p | 75.0 | 63.3 | 86.7 | 23.4 | 47.2 | 97.1 |
| miR- 4516 | 78.4 | 67.7 | 89.2 | 88.35 | 55.6 | 88.2 |

III. Attribute Selection for Variable Minimization and Selection of miRNA Combinations

In order to select miRNAs which are the best potential biomarkers, data were randomly divided by means of the caret package [M. Kuhn. “Building Predictive Models in R Using the caret Package.” J. Stat. Softw. 2008.] in R, version 3.6.1 [R. C. T. R. Foundation. “R: A Language and Environment for Statistical Computing.” vol. 2. https://www.R-project.org. 2013.] into a training set (70%) and a test set (30%).

Using the training set, 3 miRNAs were selected by means of two methods for the selection of attributes: Information Gain and Correlation-based Feature Subset Selection and these miRNAs were used to develop two logistic regression models. These logistic regression models are diagnostic classification models. In each of them the number of dependent variables was limited to two. These diagnostic classification models were validated on the test set. Selection of attributes was made using WEKA software (Waikato Environment for Knowledge Analysis Version 3.8.3). Both methods were carried out on the test set with the use of leave-one-out cross-validation (LOOV). The Information Gain method with Ranker Search method was used, which is based on the calculation of decreasing entropy by adding attributes. On this basis three best miRNAs were selected which most strongly reduce entropy: miR-1246, miR-144-3p and miR-150-5p.

Correlation-based feature selection was made with the use of the BestFirst search method (a greedy algorithm). This method is based on the results of correlation with a class and between attributes.

The strongest attributes according to this method are as follows:

miR-1246, miR-4454+miR-7975, miR-150-5p, miR-4516 and miR-144-3p.

IV. Development of a Diagnostic Classification Model and Assessment of its Quality.

To develop a diagnostic classification model, logistic regression based on miR-1246 and miR-150-5p was used. Multidimensional models make it possible to study the dependence between multiple independent variables and one dependent variable. The purpose of logistic regression is to find such a function based on variables which with highest probability properly classifies data. When such a function is found, it is possible, on the basis of independent variables, to calculate probability and classify a new subject. In this case, based on the level of a normalized number of counts of selected miRNAs from NanoString platform, which are independent variables, a logistic regression model was trained which classifies women suffering from high-grade serous ovarian cancer and women without such cancer. Below is presented an equation for the logistic regression on the basis of which a diagnostics classification model was developed:

$$P ( Y = 1 \vert {x}_{1} , {x}_{2} , … , {x}_{k} ) = \frac{{e}^{{a}_{0} + {\sum }_{i = 2}^{k} {a}_{i} {x}_{i}}}{1 + {e}^{{a}_{0} + {\sum }_{i = 2}^{k} {a}_{{i}^{x} i}}}$$

wherein
P(Y=1|x₁. x₂ . . . . . xₖ) is conditional probability that dependent variable Y will have the value of 1 for values of independent variables x₁. x₂ . . . . . xₖ
e is Euler's number≈2.718
a₀ is a constant (the point of intersection)
a₁. a₂ . . . . . aₖ are regression coefficients for individual independent variables, predictors
x₁. x₂ . . . . . xₖ are independent variables, predictors, explanatory variables.

A greater number of predictors leads to the risk of overfitting a model; that is why the present inventors have decided to use only two miRNAs in a diagnostic classification model.

V. Development of a Logistic Regression Model and Assessment of its Usefulness

A logistic regression model (binominal distribution GLM from the caret Package [M. Kuhn. “Building Predictive Models in R Using the caret Package.” J. Stat. Softw. 2008.]) was trained in a training set which included 70% of data and then it was validated on a test set (30% of data). To develop a stable model in the course of training, cross-validation, and more specifically k-fold cross-validation (K=10) was used. In this way data are divided into 10 subsets. Then, each of the subsets is sequentially used as a test set while the remaining subsets are used a training set. Thus, the analysis is conducted 10 times. In the course of developing the model this K-fold validation was conducted three times, which means that the whole training set was divided into 10 subsets three times. The results of the analysis were subsequently averaged in order to obtain one result.

The assessment of the proper classification by means of models developed on the basis of combinations of selected miRNAs made by means of ROC and AUC graphs together with the confidence interval, level of sensitivity and specificity at the cut-off point, and R² and RMSE. Calculations and graphs were made using R software, version 3.6.1 [R. C. T. R. Foundation, “R: A Language and Environment for Statistical Computing,” vol. 2, https://www.R-project.org, 2013.] and pROC packages [X. Robin et al. “pROC: An open-source package for R and S+ to analyze and compare ROC curves,” BMC Bioinformatics, vol. 12. no. 1, p. 77, March 2011], Optimal Cutpoints [M. López-Ratón, M. X. Rodríguez-Álvarez, C. Cadarso-Suárez, and F. Gude-Sampedro, “Optimalcutpoints: An R package for selecting optimal cutpoints in diagnostic tests,” J. Stat. Softw., vol. 61, no. 8, pp. 1-36, November 2014] and GraphPad Prism software, version 8. The cut-off point was calculated by Youden's method on the training set. At this point sensitivity and specificity on the test set were calculated. The table below (Table 6) presents the parameters for the trained model.

TABLE 6
Variables in the developed model for NanoString platform. a₀- constant,
a₁-coefficient for predictor x₁. a₂- coefficient for predictor x₂.

| Coefficients | a₀ | a₁ | a₂ |
|---|---|---|---|
| x1 = miR-1246 | 4.47117 | 0.07091 | −0.31985 |
| x2 = miR-150-5p | | | |

In order to be able to assess the quality of classification of the developed model, it is necessary to calculate basic quality parameters, which are presented in the table below (Table 7).

Model developed on the basis of normalized data on the miRNA expression in serum on NanoString platform:

$$P ( Y = 1 \vert miR - 1246 , miR - 150 ) = \frac{{e}^{4.47 + 0.071 * miR - 1246 - 0.32 * miR - 150 - 5 p}}{1 + {e}^{4.47 + 0.071 * miR - 1246 - 0.32 * miR - 150 - 5 p}}$$

wherein
P (Y=1|miR-1246, miR-150) is a conditional probability that the dependent variable Y will have the value of 1 for the values of independent variables: miR-1246, miR-150
e is Euler's number≈2.718.

A cut-off point within the range 0.1≤x≤0.9 gives the result for specificity (Sp) and sensitivity (S)>70% (Table 7).

TABLE 7
Cut-off point in the range 0 < x < 1 and
values of sensitivity and specificity.

| Cut-off point | Specificity (Sp) | Sensitivity (S) |
|---|---|---|
| 0.0 | 0 | 100.0 |
| 0.1 | 76.92308 | 100.0 |
| 0.2 | 76.92308 | 100.0 |
| 0.3 | 84.61538 | 100.0 |
| 0.4 | 92.30769 | 100.0 |
| 0.5 | 92.30769 | 100.0 |
| 0.6 | 92.30769 | 100.0 |
| 0.7 | 100 | 100.0 |
| 0.8 | 100 | 100.0 |
| 0.9 | 100 | 87.5 |
| 1.0 | 100 | 0 |

TABLE 8
Quality parameters for the model on the training and test set

Name

| | miRNA-1246, | miRNA-1246, |
|---|---|---|
| | miRNA-150-5p | miRNA-150-5p |
| Set | Training | Test |
| AUC | 98.6% | 100% |
| CI lower | 96.4% | — |
| Limit | | |
| CI upper | 100% | — |
| Limit | | |
| Optimal | 0.44 | 0.44 |
| cut-off point | | |
| (Youden Index) | | |
| Sensitivity | 96.4% | 100% |
| Specificity | 95.2% | 92.3% |
| R² | 0.74 | — |
| RMSE | 0.23 | — |

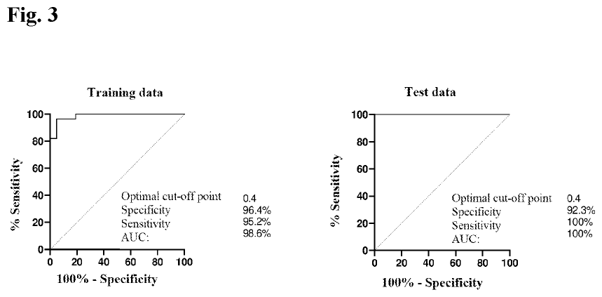

ROC curve helps to visualize the diagnostic potential of the developed models. FIG. 3. shows ROC curves, one for the training set and the other one for the test set. On each curve the cut-off point is marked, which was calculated by the Youden's statistic method (Youden's Index), and respectively, in the parentheses, is the value of sensitivity and specificity corresponding to that point.

Subsequently, a table of confusion was created (Table 9) for both models, for data which were not included while training the model. The table (Table 9) presents information about TP (true positives), TN (true negatives), FP (false positives) and FN (false negatives).

TABLE 9
Table of confusion in the test set for the
model with miRNA-1246 and miRNA-150-5p.

Actual state

| | | 0 | 1 |
|---|---|---|---|
| Prediction | 0 | 12 (TN) | 0 (FN) |
| | 1 | 1 (FP) | 8 (TP) |

VI. Validation of the Selection of Classification of miRNAs in Independent Set

To validate the potential of selected miRNA as a strong classifier of ovarian cancer on an independent cohort of patients, data from the publicly available database Gene Expression Omnibus (GEO) no. GSE106817 (https://www.ncbi.nlm.nih.gov/geo/query/acc.cgi?acc=GSE10687) were used. The study was carried out with the use of 3D-Gene Human miRNA V21_1.0.0 microarrays (Toray Industries. Inc.). Given set comprises miRNA profiles of 4046 patients, including 333 patients diagnosed with ovarian cancer, 66 women with borderline ovarian tumors, 29 with benign types of ovarian lesions, 2759 patients without a neoplasm and 859 cases with other neoplasms. The analysis was carried out with respect to data from counts of miRNA panel in serum.

From the entire data set the data of healthy subjects (n=2759) and patients with ovarian cancer (n=320) were selected. However, in order to balance the volume of groups, the data of 320 healthy persons were randomly selected. Due to the fact that the data were generated by means of another device, a model based on the expression of miR-1246, miR-150-5p was developed again, in the same way as in point V. The diagnostic potential was determined by means of ROC curve, the cut-off point was determined on the training set in the examined group. Then, it was validated on the test set, also taking into account the stage of disease. Below are presented the parameters of the trained model.

TABLE 10
Variables in the developed model for microarray.

| | | Coefficient of | Coefficient of |
|---|---|---|---|
| | | predictor x₁ | predictor x₂ |
| Coefficients | Constant a₀ | (a₁) | (a₂) |
| x₁ = miR-1246 | −2.57007 | 0.05412 | 0.49059 |
| x₂ = miR-150-5p | | | |

A logistic regression model was developed on the basis of normalized data on the miRNA expression in serum on Affymetrix platform (microarray) with the use of the following formula:

$$P ( Y = 1 \vert miR - 1246 , miR - 150 ) = \frac{{e}^{- 2.57 + 0.054 * miR - 1246 + 0.49 * miR - 150 - 5 p}}{1 + {e}^{- 2.57 + 0.054 * miR - 1246 + 0.49 * miR - 150 - 5 p}}$$

wherein
P (Y=1|miR-1246, miR-150) is a conditional probability that the dependent variable Y will have the value equal to 1 for the values of the independent variables miR-1246, miR-150
e is Euler's number≈2.718.

A cut-off point within the range of 0.4≤x≤0.7 gives the result for specificity (Sp) and sensitivity (S)>70% (Table 11).

TABLE 11
Cut-off point within the range of 0 < x <
1 and values of sensitivity and specificity.

| Cut-off point | Specificity (Sp) | Sensitivity (S) |
|---|---|---|
| 0.0 | 0 | 100.0 |
| 0.1 | 35.41667 | 92.70833 |
| 0.2 | 47.91667 | 91.66667 |
| 0.3 | 65.62500 | 90.62500 |
| 0.4 | 77.08333 | 87.50000 |
| 0.5 | 85.41667 | 83.33333 |
| 0.6 | 89.58333 | 78.12500 |
| 0.7 | 94.79167 | 70.83333 |
| 0.8 | 95.83333 | 53.12500 |
| 0.9 | 98.95833 | 27.08333 |

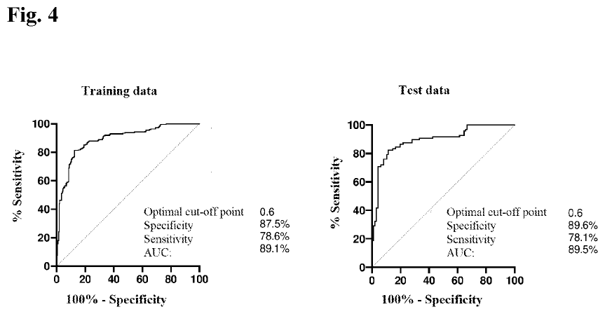

FIG. 4 shows ROC and AUC curves obtained for the trained model on the training and test set. The cut-off point is marked on the curve, AUC and the confidence interval in the parentheses are also given.

Subsequently, a table of confusion was created on the test set for the diagnostic classification model for the microarray with miR-1246 and miR-150-5p on the basis of data from the test set. Table 12 shows information about the number of true positive (TP), true negative (TN), false positive (FP) and false negative (FN) results.

TABLE 12
Table of confusion in the test set for the model for data
from the microarray with miRNA-1246, miRNA-150-5p.

Actual state

| | | 0 | 1 |
|---|---|---|---|
| Prediction | 0 | 182 (TP) | 37 (FN) |
| | 1 | 42 (FP) | 187 (TF) |

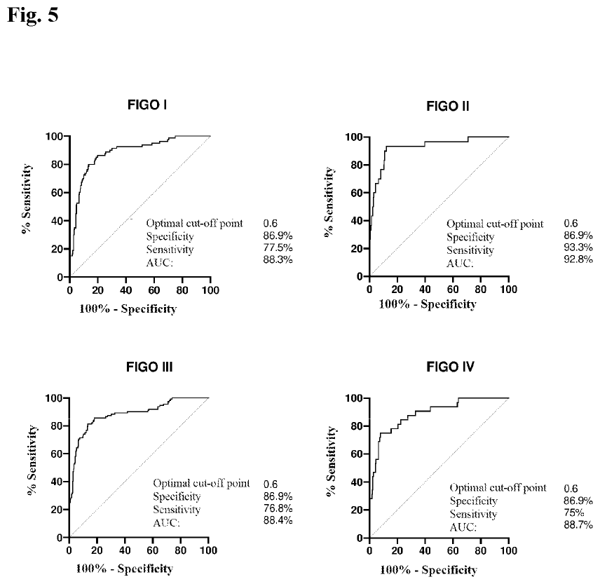

FIG. 5 shows ROC curves for the trained classification model based on logistic regression and the calculated AUC with a confidence interval for different stages of ovarian cancer according to FIGO. For each of the stages the sensitivity and specificity of the model was calculated at specific cut-off point. Table 13 contains a summary of results concerning the quality parameters for the developed model based on the two selected miRNAs (miR-1246 and miR-150-5p). Since information about the stage of development of the disease (FIGO) was available in the validation data, the table also contains information about the group size and the classification result for individual stages of disease.

TABLE 13
Results of quality assessment of the
model with the use of external data

Name

| | miRNA-1246. | miRNA-1246. |
|---|---|---|
| | miRNA-150-5p | miRNA-150-5p |
| Set | Training | Test |
| AUC (95% CI) | 89.1% | 89.5% |
| CI lower limit | 86.1% | 84.8% |
| CI upper limit | 92.1% | 94.1% |
| Cut-off point | 0.56 | 0.56 |
| Sensitivity | 81.2% | 82.3% |
| Specificity | 87.1% | 87.5% |
| FIGO I (n = 80) | | |
| AUC (95% CI) | — | 88.3% |
| FIGO II (n = 30) | | |
| AUC (95% CI) | — | 92.8% |
| FIGO III (n = 112) | | |
| AUC (95% CI) | — | 88.4% |
| FIGO IV (n = 32) | | |
| AUC (95% CI) | — | 88.7% |

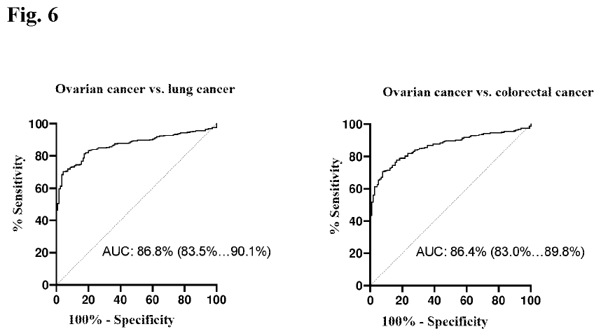

Additionally, publically available data VI (data from: https://www.ncbi.nlm.nih.gov/geo/query/acc.cgi?acc=GSE106817) comprising also the data on the miRNA expression in serum of patients with other cancers were used to create ROC curves and calculate AUC with a confidence interval on the basis of the developed diagnostic classification model. The results for the trained model are presented in FIG. 6. It can be seen that the diagnostic classification model is able to property differentiate the subjects suffering from ovarian cancer from the subjects suffering from lung cancer or subjects suffering from colorectal cancer.

VII. Diagnostic Classification Model for RT-qPCR Method

In order to develop a diagnostics classification model capable of application in practice, a reverse transcription and real-time polymerase chain reaction (RT-qPCR) was used to assess the miR-1246 and miR-150-5p expression level. The test included an enlarged group of patients (n=88), including 42 patients with ovarian cancer and 46 patients in the control group (Table 14).

TABLE 14
Characteristics of patients whose samples were
included in the tests by RT-qPCR method

| | | Ovarian |
|---|---|---|
| | Control | cancer |
| Number of patients | 46 | 42 |

| | Mean ± SD | Min | Max | p value |
|---|---|---|---|---|
| Age at diagnosis | | | | |
| (years) | | | | |
| Healthy | 59 + 7 | 43 | 77 | 0.08 |
| Diseased | 63 + 13 | 38 | 86 | |
| BMI (weight/height²) | | | | |
| Healthy | 27 + 4 | 21 | 39 | 0.7 |
| Diseased | 28 + 5 | 19 | 42 | |

| | FIGO I | FIGO II | FIGO III-IV | N/A |
|---|---|---|---|---|
| Number of cases | 3 | 1 | 34 | 4 |

A reactant kit mirCURY LNA RT Kit (Qiagen. Germany) was used for the reverse transcription reaction, whereas for the proper real-time PCR, miRCURY LNA SYBR Green PCR Kit (Qiagen. Germany) was used. The reaction was conducted with the use of the following starters: for miR-1246, MIMAT0005898: 5′ AAUGGAUUUUUGGAGCAGG; for miR-150-5p, MIMAT0000451: 5′UCUCCCAACCCUUGUACCAGUG. Two reference miRNAs were used: miR-103-3p, MIMAT0000101: AGCAGCAUUGUACAGGGCUAUGA and miR-199b-5p, MIMAT0000263: The temperature profile of RT-qPCR was as follows: 2 min at 95° C. and 50 cycles: 10 s at 95° C. and 60 s at 56° C. Reaction efficiency for each pair of the starters was calculated by preparing a series of template dilutions. Subsequently, PCR thresholds cycles (Cₜ) of the tested miRNAs and reference miRNA were determined for the tested samples and the calibrator. Relative expression level of the tested miRNAs was determined according to the formula:

$$R = \frac{{( {E}_{tested gene} )}^{Δ {C}_{p} tested gene ( contro1 - test )}}{{( {E}_{control gene} )}^{Δ {C}_{p} control gene ( contro1 - test )}}$$

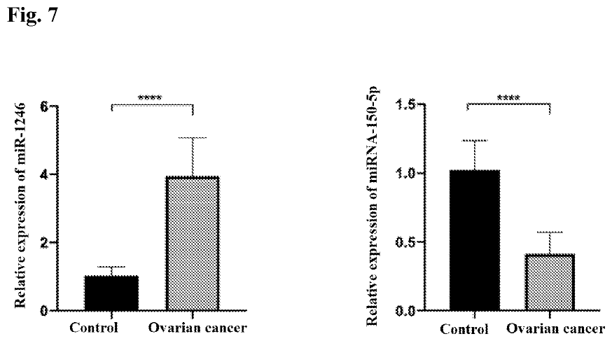

It was demonstrated that miR-1246 exhibits increased expression in serum of women with ovarian cancer, as opposed to miR-150-5p the expression of which in women suffering from ovarian cancer is reduced. The results are presented in FIG. 7.

Based on the normalized RT-qPCR data, a diagnostic classification model (binominal distribution GLM form the caret Package [M. Kuhn. “Building Predictive Models in R Using the caret Package.” J. Stat. Softw. 2008.]) was developed, which was trained on a training set comprising 70% of data and validated on a test set (30% of data). In order to develop a stable diagnostic classification model, a leave-one-out cross-validation method was used in the course of training. A specific number of subsets were created such that each patient was included in the test group once whereas the remaining subjects were in the training set and a logistic regression model was also created on the basis of each of the subsets. The results of the analyses were subsequently averaged in order to obtain one most optimal result. The assessment of the proper classification was made by means of ROC curves, determining AUC together with the confidence interval, level of sensitivity and specificity at the cut-off point, and R² and RMSE. Calculations and graphs were made using R software, version 3.6.1 [R. C. T. R. Foundation. “R: A Language and Environment for Statistical Computing.” vol. 2. https://www.R-project.org. 2013.] and Proc packages [X. Robin et al. “pROC: An open-source package for R and S+ to analyze and compare ROC curves.” BMC Bioinformatics, vol. 12, no 1, p. 77, March 2011]. Optimal Cutpoints [M. López-Ratón. M. X. Rodríguez-Álvarez. C. Cadarso-Suárez. and F. Gude-Sampedro. “Optimalcutpoints: An R package for selecting optimal cutpoints in diagnostic tests.” J. Stat. Softw., vol. 61, no. 8, pp. 1-36, November 2014.]. The cut-off point was calculated by means of the Youden's method on the training set. At this point sensitivity and specificity were calculated on the test set. (Table 15).

TABLE 15
Variables in the developed model for data from RT-qPCR. a₀- constant,
a₁-coefficient of predictor x₁, a₂- coefficient of predictor x₂.

| Coefficients | a₀ | a₁ | a₂ |
|---|---|---|---|
| x₁ = miR-1246 | 55.160 | −1.616 | 4.277 |
| x₂ = miR-150-5p | | | |

A diagnostic classification model was developed on the basis of normalized data on the miRNA expression level in serum, obtained by RT-qPCR method with the use of the formula:

$$P ( Y = 1 \vert miR - 1246 , miR - 150 ) = \frac{{e}^{55.16 - 1.616 * miR - 1246 + 4.277 * miR - 150 - 5 p}}{1 + {e}^{55.16 - 1.616 * miR - 1246 + 4.277 * miR - 150 - 5 p}}$$

wherein
P (Y=1|miR-124, miR-150) is a conditional probability that the dependent variable Y will have the value equal to 1 for the values of independent variables miR-1246, miR-150
e is Eurel's number≈2.718.

A cut-off point within the range of 0.1<x≤0.8 gives the result for sensitivity (S) and specificity (Sp) in the test set>85% (Table 16).

TABLE 16
Cut-off points within the range of 0 < x ≤
0.9 and values of sensitivity and specificity.

| Cut-off point | Specificity | Sensitivity |
|---|---|---|
| 0.0 | 0 | 100.0 |
| 0.1 | 91.66667 | 100.0 |
| 0.2 | 91.66667 | 100.0 |
| 0.3 | 91.66667 | 100.0 |
| 0.4 | 91.66667 | 92.85714 |
| 0.5 | 91.66667 | 92.85714 |
| 0.6 | 91.66667 | 92.85714 |
| 0.7 | 91.66667 | 92.85714 |
| 0.8 | 91.66667 | 85.71429 |
| 0.9 | 91.66667 | 78.57143 |

Basic quality parameters were also calculated in order to assess the quality of the diagnostic classification model, which are presented in Table 17 below.

TABLE 17
Quality parameters for models in the training and test set

Name

| | miR-1246. | miR-1246. |
|---|---|---|
| | miR-150-5p | miR-150-5p |
| Set | Training | Test |
| AUC | 99.7% | 94.6% |
| CI lower | 99.0% | 83.9% |
| Limit | | |
| CI upper | 100% | 100% |
| Limit | | |
| Optimal | 0.2 | — |
| cut-off point | | |
| (Youden Index) | | |
| Sensitivity | 96.4% | 100% |
| Specificity | 94.1% | 91.7% |

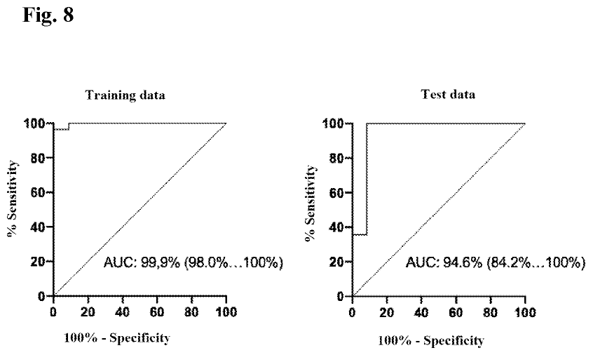

ROC curve helps to visualize the diagnostic potential. FIG. 8 shows ROC curves for the trained model, one curve is for the training set and the other one is for the test set. On each curve the cut-off point is marked, which was calculated by the J Youden's statistic method, and sensitivity and specificity corresponding to that point.

Subsequently, a table of confusion (Table 18) was created for data which were not included in the course of training the model. Table 16 shows information about the number of true positive (TP), true negative (TN), false positive (FP) and false negative (FN) results after classification.

TABLE 18
Table of confusion in the test set for the
model with miRNA-1246 and miRNA-150-5p.

Actual state

| | | 0 | 1 |
|---|---|---|---|
| Prediction | 0 | 11 (TP) | 0 (FN) |
| | 1 | 1 (FP) | 14 (TN) |

1. A diagnostic panel of miRNA biomarkers comprising miR-1246 and miR-150-5p.

2. (canceled)

3. A method for in vitro diagnosis of ovarian cancer in a subject, characterized in that it comprises the following steps:
i) determining the expression level of a panel of two miRNA biomarkers: miR-1246 and miR-150-5p in a sample from a subject and
ii) comparing the levels determined in step i) with the expression levels of miR-1246 and miR-150-5p in a subject without ovarian cancer, wherein the comparison provides a diagnostic indicator determining whether the subject has ovarian cancer.

4. The method according to claim 3, characterized in that an increase in the miR-1246 expression level relative to the miR-1246 expression level in a subject without ovarian cancer and a decrease in the miR-150-5p expression level relative to the miR-150-5p expression level in a subject without ovarian cancer indicate ovarian cancer in the subject.

5. The method according to claim 3, characterized in that the expression levels are normalized levels.

6. The method according to claim 3, characterized in that a comparison of the expression levels is made with the use of a diagnostic classification model which classifies a sample as a sample from a subject with ovarian cancer or a sample from a subject without ovarian cancer.

7. The method according to claim 6, characterized in that a diagnostic classification model compares the expression levels with the use of a data set comprising data on the expression level of a panel of miRNA markers comprising miR-1246 and miR-150-5p.

8. The method according to claim 1, characterized in that the expression level of a panel of miRNA biomarkers is determined with the use of a method for measuring the expression selected from quantitative reverse transcription and polymerase chain reaction (qPCR).

9. (canceled)

10. (canceled)

11. (canceled)

12. (canceled)

13. (canceled)

14. The method according to claim 3, characterized in that a sample is a serum sample.

15. The method according to claim 3, characterized in that ovarian cancer is high-grade serous ovarian cancer.

16. (canceled)

17. (canceled)

18. A method of treating ovarian cancer in a subject comprising monitoring response to ovarian cancer treatment by
i) determining the expression level of a panel of two miRNA biomarkers: miR-1246 and miR-150-5p in a sample from a subject after ovarian cancer treatment, and
ii) comparing the levels determined in step i) with the expression levels of miR-1246 and miR-150-5p in the subject before ovarian cancer treatment, wherein the comparison provides an indicator of the subject's response to the ovarian cancer treatment for adjustment of the ovarian cancer treatment.

19. (canceled)

20. (canceled)

21. A test for in vitro diagnosis of ovarian cancer or for determining recurrence of ovarian cancer after competed ovarian cancer treatment, characterized in that it comprises means for quantitative determination of the expression level of a panel of two miRNA biomarkers: miR-1246 and miR-150-5p and instructions for carrying out the method according to claim 3.

22. The test for the diagnosis according to claim 21, characterized in that as means for the quantitative determination of the expression of miRNA biomarkers: miR-1246 and miR-150-5p it comprises reactants and primers for amplification in RT-qPCR reaction.

23. The test for the diagnosis according to claim 22, characterized in that it further comprises means for quantitative determination of the expression of a reference miRNA, preferably a panel of miR-103-3p and/or miR-199b-5p.

24. The method of claim 8, wherein the expression level of a panel of miRNA biomarkers is determined with the use of a real-time analysis of product quantity (RT-qPCR), NanoString or microarray method.

25. The method according to claim 24, characterized in that a diagnostic classification model uses for the classification an optimal cut-off point selected from: 0.1-0.8 in the case of expression measurement by RT-qPCR method; 0.3-0.9 in the case of expression measurement by NanoString method and the value of in the case of expression measurement with the use of microarray.

26. The method according to claim 25, characterized in that the RT-qPCR method is used for determination of the miRNA expression level.

27. The method according to claim 26, characterized in that the expression level of a reference miRNA, preferably miR-103-3p and/or miR-199b-5p, is used for normalization of results.

28. The method according to claim 27, characterized in that normalization of results is obtained with the use of the formula:
deltaCt=(Ct miR-1246/miR-150-5p-Ct miR-103-3p).
wherein
deltaCt is a change in the value of threshold cycle
Ct is the value of threshold cycle.

29. The method according to claim 28, characterized in that the obtained normalized value of deltaCt after comparison with a cut-off point classifies a subject from whom a sample was taken as a subject with or without ovarian cancer.
